SEQUENCE LISTING (1 sequence)

SEQ ID NO: 1 - protein, 332 aa, Homo sapiens
VKVGVDGFGRIGRLVTRAAFNSGKVDIVAINDPFIDLHYMVYMFEYDSTHGKFHGTVKAEDGKLVIDGRAITIFQERDPANIKWGDAGTAYVVESTGVFTTMEKAGAHLKGGAKRVIISAPSADAPMFVMGVNHEKYDNSLKIVSNASCTTNCLAPLAKVIHDHFGIVEGLMTTVHAITATQKTVDGPSGKLWRDGRGAAQNIIPASTGAAKAVGKVIPELDGKLTGMAFRVPTPNVSVVDLTCRLEKPAKYDDIKKVVKQASEGPLKGILGYTEDQVVSCDFNDSTHSSTFDAGAGIALNDHFVKLISWYDNEFGYSNRVVDLMVHMASKE

(19) United States
(12) Patent Application Publication
(10) Pub. No.: US 2011/0159523 A1
(43) Pub. Date: Jun. 30, 2011

(54) N-TERMINAL SPECIFIC CHEMICAL LABELING FOR PROTEOMICS APPLICATIONS

(75) Inventors: Parag Mallick, Los Angeles, CA (US); Jeremy C. Collette, Los Angeles, CA (US)

(73) Assignee: CEDARS-SINAI MEDICAL CENTER, Los Angeles, CA (US)

(21) Appl. No.: 12/666,622

(22) PCT Filed: Jun. 27, 2008
(86) PCT No.: PCT/US08/68672

§ 371 Date: Mar. 18, 2011

Related U.S. Application Data
(60) Provisional application No. 60/946,502, filed on Jun. 27, 2007.

(51) Int. Cl. G01N 30/72; C12M 1/40

(52) U.S. Cl. 435/7.72; 435/288.6; 435/287.1

(57) ABSTRACT

Described herein is a method that may be used in various applications, such as drug development, medical diognosis and gene/protein therapy. In one embodiment, the subject matter discloses an effective method for identification and quantification of large sets of proteins/peptides in vitro and cells in vivo.

### FIELD OF THE SUBJECT MATTER

The present subject matter relates to methods for identification and quantification of large sets of proteins and peptides in vitro and cells in vivo.

### BACKGROUND OF THE SUBJECT MATTER

All publications herein are incorporated by reference to the same extent as if each individual publication or patent application was specifically and individually indicated to be incorporated by reference. The following description includes information that may be useful in understanding the present subject matter. It is not an admission that any of the information provided herein is prior art or relevant to the presently claimed subject matter, or that any publication specifically or implicitly referenced is prior art.

Proteomics is the large-scale study of proteins, focusing particularly on protein structures and functions. Proteins are vital parts of living organisms, as they are the main components of the physiological metabolic pathways of cells. Proteomics is often considered the next step in the study of biological systems, after genomics, and is much more complicated than genomics. The complexity behind proteomics is attributed to the fact that, while an organism's genome is rather constant, a proteome differs from cell to cell and constantly changes through its biochemical interactions with the genome and the environment. Thus an organism has radically different protein expression in different parts of its body, different stages of its life cycle and different environmental conditions. This increased complexity derives from mechanisms such as alternative splicing, protein modification (glycosylation, phosphorylation) and protein degradation.

The scientific community is very much interested in proteomics because it gives a much better understanding of an organism than genomics. First, the level of transcription of a gene gives only a rough estimate of its level of expression into a protein. An mRNA produced in abundance may be degraded rapidly or translated inefficiently, resulting in a small amount of protein. Second, many proteins experience post-translational modifications that profoundly affect their activities; for example some proteins are not active until they become phosphorylated. Methods such as phosphoproteomics and glycoproteomics are used to study post-translational modifications. Third, many transcripts give rise to more than one protein, through alternative splicing or alternative post-translational modifications. Finally, many proteins form complexes with other proteins or RNA molecules, and only function in the presence of these other molecules.

Since proteins play a central role in the life of an organism, proteomics is instrumental in the discovery of biomarkers, such as markers that indicate a particular disease. With the completion of a rough draft of the human genome, many researchers are turning their attention to how genes and proteins interact to form other proteins. A surprising finding of the Human Genome Project is that there are far fewer protein-coding genes in the human genome than proteins in the human proteome (20,000 to 25,000 genes vs. >500,000 proteins). The human body may even contain more than 2 million proteins, each having different functions. The protein diversity is thought to be due to alternative splicing and post-translational modification of proteins. The discrepancy implies that protein diversity cannot be fully characterized by gene expression analysis, thus proteomics is useful for characterizing cells and tissues.

The identification of proteins, their functions and interactions is a great challenge for the scientific community. Accurate and reproducible quantitative measurement of levels of clinically relevant proteins has been hampered by the vast complexity and dynamic range of protein samples. Traditional methods for determining relative protein expression levels are laborious, require high expertise for reproducibility, fail to interrogate proteins outside limited pH and size ranges, possess limited resolution, and have unacceptably low sensitivity for clinical applications. Although proteomics platforms have overcome some of these limitations and been impressively applied as tools for answering biologic questions, major challenges must still be overcome before they can be widely applied to important clinical questions, like cancer prognosis and diagnosis. One such challenge is the high-throughput, reproducible, comparative quantification and identification of large numbers of clinically relevant peptides and proteins. Dynamic range has been cited as a significant confound to clinical application of proteomic platforms; the dynamic range of protein abundances in yeast and other model organisms, has been estimated at six orders of magnitude whereas the dynamic range of mammals has been estimated to be tremendous and beyond the reach of current proteomic platforms.

Several methods are available for protein identification. The most recent methods include protein microarrays, immunoaffinity chromatography, mass spectrometry, and combinations of experimental methods such as phage display and computational methods.

Bottom-up mass spectrometry (MS) has emerged as the best approach for identification of proteins, and utilizes an enzyme (e.g. trypsin) to digest proteins into small peptides. In many cases, the sequence of a peptide fragment is sufficient to identify the protein it came from. An advantage of the bottom-up MS approach is that peptides are much easier to fractionate and identify than whole proteins; however, a disadvantage is that each protein digested by trypsin gives rise to 50 peptides, on average, resulting in a huge increase in the complexity of the mixture.

Researchers have exploited the difference in the pKa of the amide proton between lysines and the N-terminus by raising the pH of the reaction mixture to favor incorporation of a label at the N-terminus. However, this method has limited specificity. Another approach is to block lysine residues by guanidylation before adding a label. This method achieves improved specificity, but like the other method, cannot be used to perform labeling in cells, making it unsuitable for an important class of proteomic experiments. Enzyme mediated transamination of single amino acids is also known; however, these reactions lack the specificity to target the N-terminal amine independently of epsilon-amines of lysines, or they use enzymatic methods that are less flexible.

As evidenced above, the current methods for protein identification are plagued by lack of reproducibility, throughput, dynamic range, or all three. Accordingly, there is a need in the art to develop a novel method to identify and quantify large sets of proteins/peptides in vitro and cells in vivo.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a general reaction scheme for copper/glyoxylate mediated transamination of proteins and peptides in accordance with an embodiment of the present subject matter. The N-terminal alpha-amine is transformed into an alpha-ketoamide. The alpha-keto amide is then the aminooxy reactive species that forms the oxime.

FIG. 2 depicts a general reaction scheme of PLP mediated transamination followed by oxime formation in accordance with an embodiment of the present subject matter. In this example, the protein is N-terminally biotinylated via reaction of the transamination product, the alpha-ketoamide, with aminoxy biotin.

FIG. 3A depicts results of protein labeling experiments in accordance with an embodiment of the present subject matter. In this example, the Figure depicts MALDI-MS results of Ubiquitin labeled protein mixture, with the top panel depicting the control and the bottom panel depicting the benzyl labeled. Mass spectrometry measures mass of analyte, and labeling proteins adds mass to analyte per adduct. Single adduct adds mass equal to one label. Multiple adducts add mass equal to multiple labels. The label mass equals 106 daltons and microcon centrifugal filter device is used for a purification method.

FIG. 3B depicts results of protein labeling experiments in accordance with an embodiment of the present subject matter. In this example, the Figure depicts MALDI-MS results of Myoglobin labeled protein mixture, with the top panel depicting the control and the bottom panel depicting the benzyl labeled. Mass spectrometry measures mass of analyte, and labeling proteins adds mass to analyte per adduct. Single adduct adds mass equal to one label. Multiple adducts add mass equal to multiple labels. The label mass equals 106 daltons and microcon centrifugal filter device is used for a purification method.

FIG. 3C depicts results of protein labeling experiments in accordance with an embodiment of the present subject matter. In this example, the Figure depicts MALDI-MS results of Cytochrome C labeled protein mixture. The top panel depicts the control, where acetylated in vivo and should be mostly unlabeled. The bottom panel depicts the benzyl labeled. Mass spectrometry measures mass of analyte, and labeling proteins adds mass to analyte per adduct. Single adduct adds mass equal to one label. Multiple adducts add mass equal to multiple labels. The label mass equals 106 daltons and microcon centrifugal filter device is used for a purification method.

FIG. 4 depicts results of peptide labeling experiments in accordance with an embodiment of the present subject matter. The Figure depicts results of experiments to N-terminally label proteins, digested with trypsin, and uses MS sequencing methods to validate that the label is on the N-terminal peptide, by MS/MS of N-terminally labeled GAPDH (SEQ. ID. NO. 1).

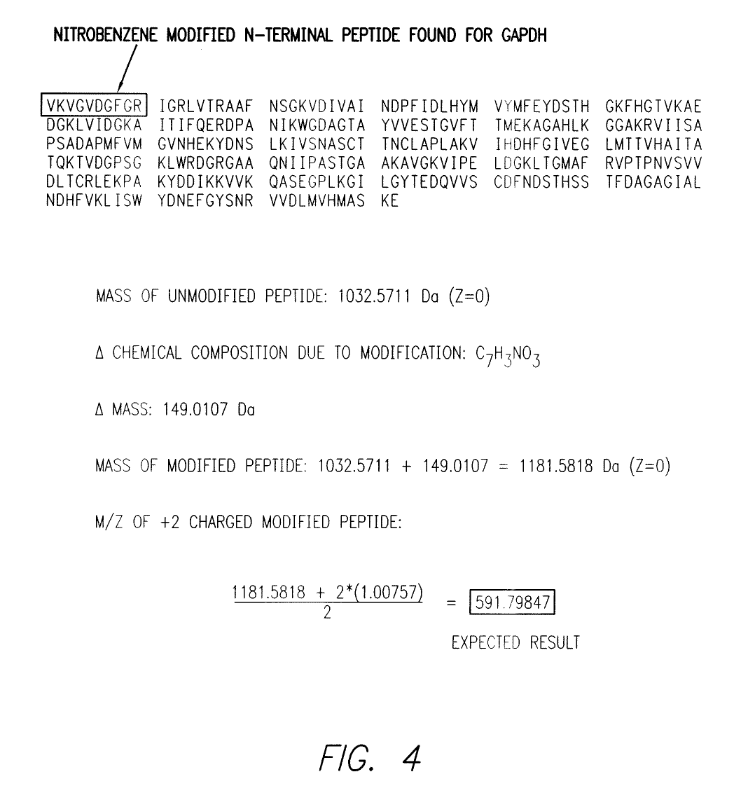

FIG. 5A depicts results of peptide labeling experiments in accordance with an embodiment of the present subject matter. The Figure depicts results of experiments to N-terminally label proteins, digested with trypsin, and uses MS sequencing methods to validate that the label is on the N-terminal peptide, with mass as expected for labeled N-terminal peptide.

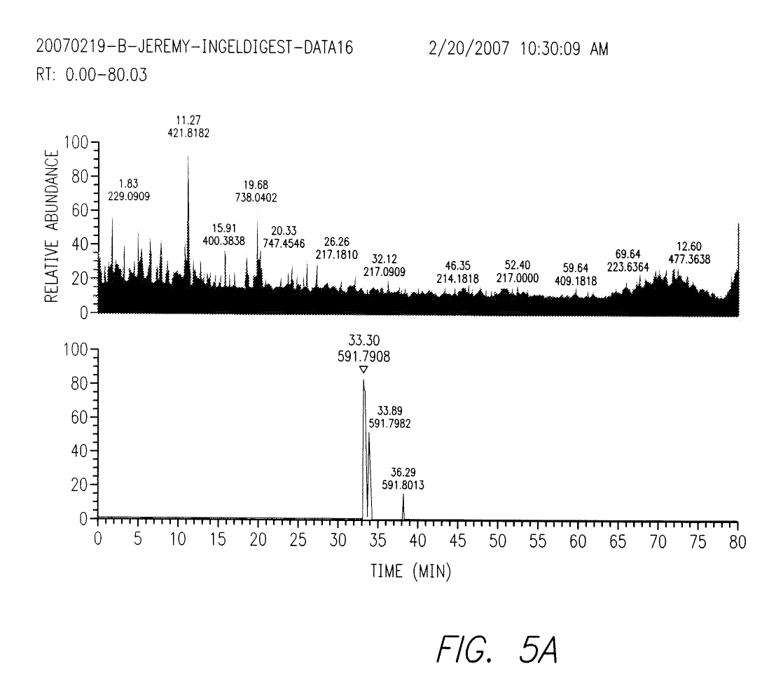

FIG. 5B depict results of peptide labeling experiments in accordance with an embodiment of the present subject matter. The Figure depicts results of experiments to N-terminally label proteins, digested with trypsin, and uses MS sequencing methods to illustrate the expected mass for the N-terminal labeled peptide.

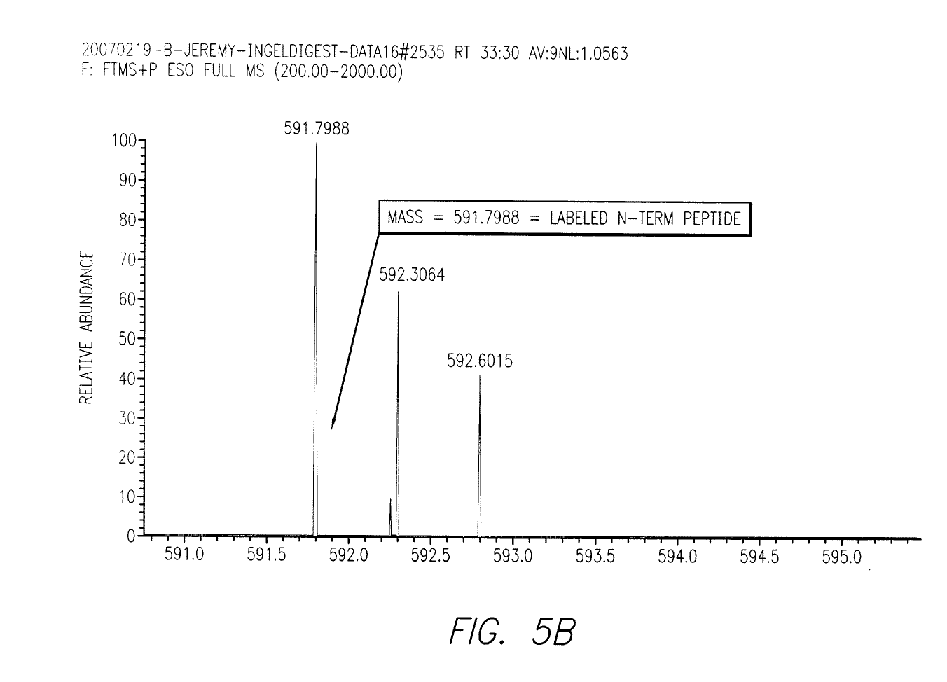

FIG. 6 depicts results of peptide labeling experiments in accordance with an embodiment of the present subject matter. The Figure depicts results of experiments to N-terminally label proteins, digested with trypsin, and uses MS sequencing methods to validate that the label is on the N-terminal peptide, with the peptide sequence of a nitrobenzene modified N-terminal peptide for GAPDH.

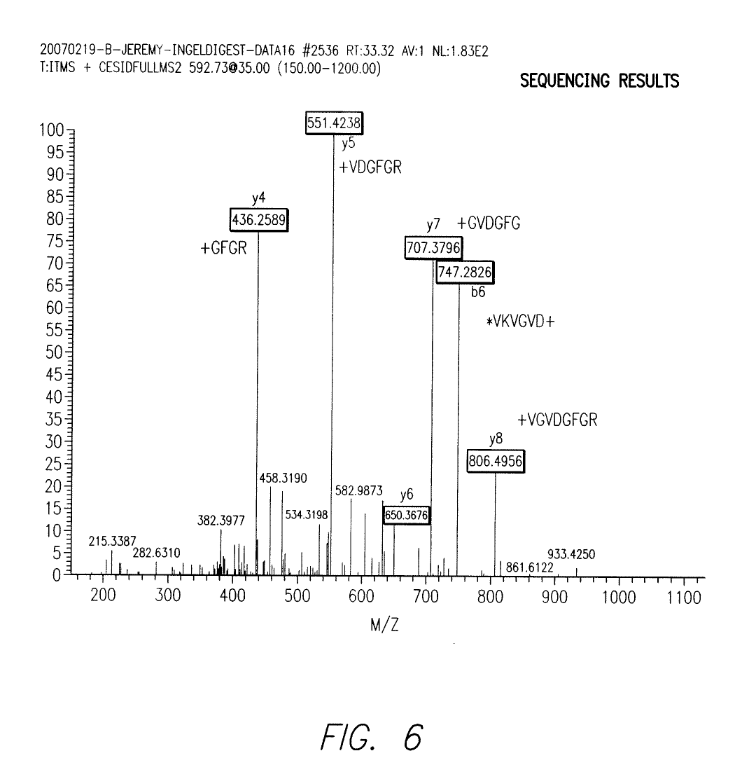

FIG. 7 depicts N-terminal biotin labeling of a protein mixture in accordance with an embodiment of the present subject matter. The Figure depicts the results of experiments to label the N-terminal of protein with biotin, perform PAGE, stain proteins with coomassie, and then run western with avidin-hrp, demonstrating the biotinylation of mixture of proteins.

FIG. 8 depicts a flow chart detailing the system for identifying a protein or peptide by mass spectrometry in accordance with an embodiment of the present subject matter.

### DETAILED DESCRIPTION OF THE SUBJECT MATTER

All references cited herein are incorporated by reference in their entirety as though fully set forth. Unless defined otherwise, technical and scientific terms used herein have the same meaning as commonly understood by one of ordinary skill in the art to which this subject matter belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present subject matter. Indeed, the present subject matter is in no way limited to the methods and materials described.

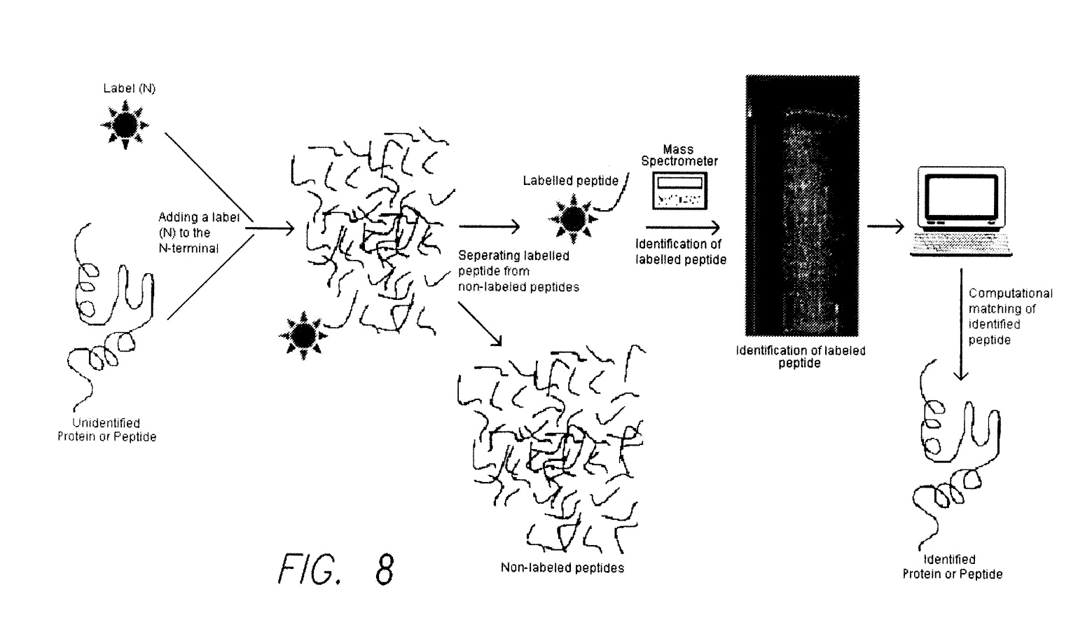

The inventive subject matter is a chemical process that adds an “affinity” label to the N-terminus of each protein in the mixture for enhanced protein and peptide identification and quantification. After digestion, exactly one peptide per protein (the one containing the original N-terminus of the protein) will carry the label. These labeled peptides can be separated from the unlabeled peptides by running the mixture over a fixed support containing an immobilized reagent with selective affinity for the label. The captured peptides are eluted from the support and then analyzed, as before, by mass spectrometry. The mass spectrometrical analysis produces a list of molecular weights which is often called a peak list. The peptide masses undergo computational analysis where the results are now compared to databases such as Swissprot Genbank which contain protein sequence information. Software programs cut all these proteins into peptides with the same enzyme used in the chemical cleavage (for example trypsin). The absolute mass of all these peptides is then theoretically calculated, and a comparison is made between the peak list of measured peptide masses and all the masses from the calculated peptides. The results are statistically analyzed and possible matches are returned in a results table. A flow chart for the process described above can be found in FIG. 8.

The resulting captured peptides have significantly reduced complexity, dramatically facilitating both identification of peptides and subsequent matching to proteins. Furthermore, the N-Terminal labeling of proteins can be used to reduce the sample complexity associated with bottom-up mass spectrometry, while still allowing the advantages of peptide analysis.

In addition, N-Terminal labeling of peptides (whether before or after digestion) can simplify peptide sequencing by tandem mass spectrometry. As explained herein, the sequence of a peptide is often sufficient for identifying the protein it came from. Tandem mass spectrometry involves breaking the peptide into fragments and using the identities of the fragments to infer the sequence. If the label is chosen judiciously, fragments containing the label can be detected by virtue of their mass alone. Therefore, the subset of fragments containing the N-terminus can be identified, making the determination of sequence much simpler.

In addition to simplifying the complexity of the protein or peptide mixture to be characterized, the inventive subject matter includes methods of labeling proteins/peptides with atoms demonstrating significant mass defect. Mass defect is the difference between the fractional and nominal mass of an atom. In other words, an atomic mass defect is the difference between the sum of the masses of the subatomic particles minus the mass observed. Atoms like bromine and selenium have large mass defects at around 0.1 unified atomic mass unit or Dalton. The N-terminal labeling of proteins with atoms demonstrating a significant unified atomic mass unit, shifts the mass of peptide out of expected detection regions into unpopulated mass regions making their identification easier. Additionally, certain atoms with a significant unified atomic mass unit, such as bromine, have a very distinct isotopic envelope. Combining the pattern recognition of the bromine isotopic envelope with the mass shift out of expected detection regions, additionally increases the probability of accurate peptide and protein identification.

N-Terminal labeling of peptides and proteins is possible because of the unique occurrence of the alpha-amine group only at the N-terminus. However, specific labeling of the N-terminus is confounded by the fact that every lysine residue in a peptide or protein contains an epsilon-amine group that has similar reactivity. Therefore, care must be taken to develop a chemical process that can distinguish these groups. By employing N-Terminal labeling of proteins in complex mixtures with synthetic tags, the complexity of the mixture may be reduced by up to 50-fold.

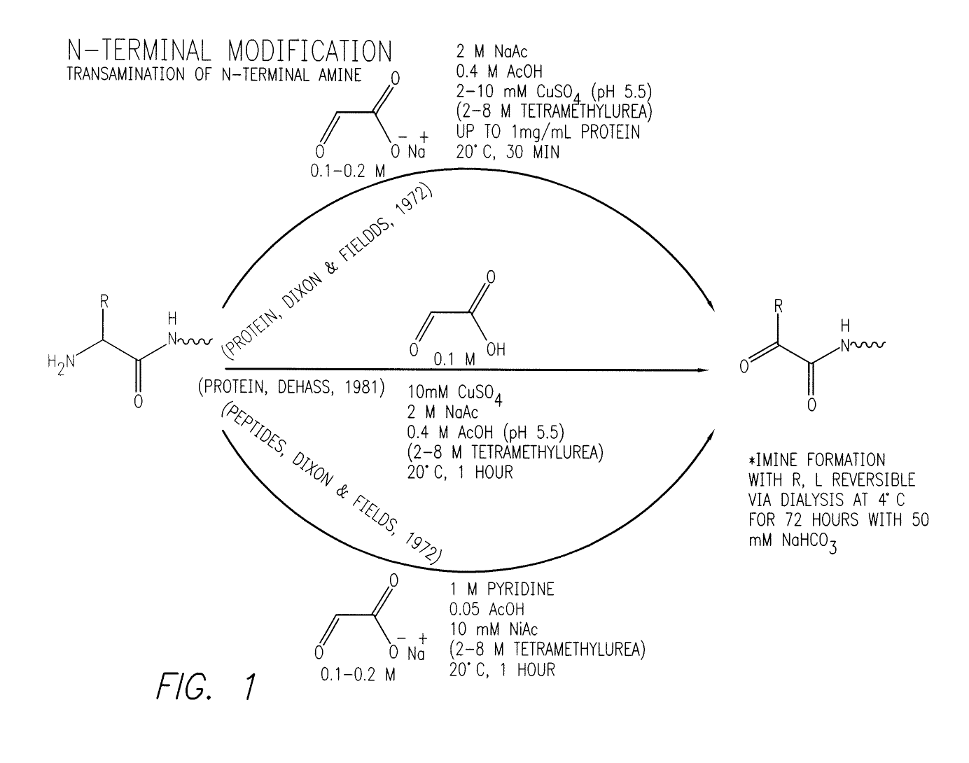

In one embodiment, depicted in FIG. 1, the present subject matter provides methods of conjugating a molecule to the N-termini of a peptide and/or protein by the following steps: transamination and transformation of a peptide and/or protein N-terminal alpha-amine to an alpha-ketoamide; the reactive intermediate is then condensed with an alkoxyamine to form an oxime. The oxime can be further stabilized by reduction to an amine with sodium cyanoborohydride.

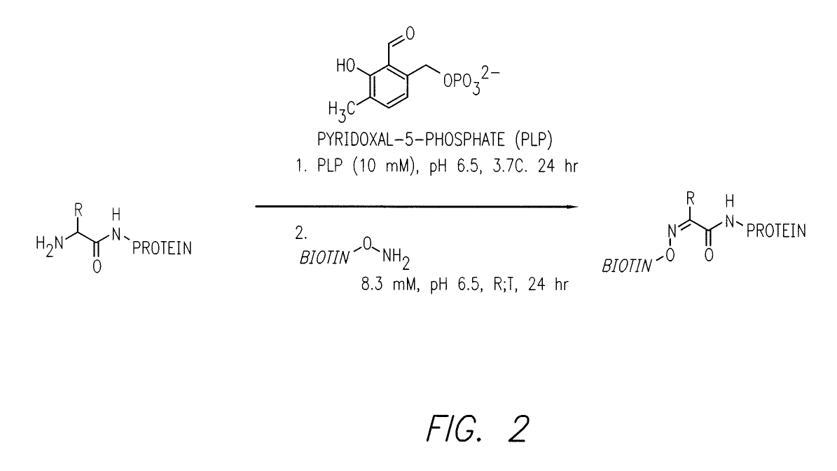

In another embodiment, depicted in FIG. 2, the chemical process involves a first step of transaminating N-terminal (alpha-amine transformation to alpha-ketoamide) with pyridoxal-5-phosphate and copper/glyoxylate, followed by reacting the product of the first step with alkoxyamine (also known as hydroxylamine and aminooxy) reagents to form an oxime and then optionally reducing the oxime to a secondary amine with sodium cyanoborohydride to stabilize the conjugate.

In yet another embodiment, the chemical process engages the first step of transaminating the N-terminal with coper/glyoxylate producing an alpha-amine. The N-terminal alpha amine is transformed into an alpha-ketoamide, which is then the aminooxy reactive species that forms the oxime.

In another embodiment, the protein is N-terminally biotinylated via reaction of the transamination product (pyridoxal-5-phosphate), the alpha-ketoamide, with aminoxy biotin.

In one embodiment, the present subject matter provides methods of determining the identity, quantity, topology, degradation and/or turnover of cell surface protein by N-terminal amine specific labeling of cell surface protein and determining the presence and/or absence of the label. In another embodiment, the present subject matter provides methods of cell and/or analyte detection, quantification, growth analysis and/or viability control by N-terminal amine specific labeling of a cell surface and determining the presence and/or absence of the label. In another embodiment, the cell surface is labeled with fluorophore and/or quantum dot. In another embodiment, the label is identified by flow cytometry.

In one embodiment, the present subject matter provides methods of proteomic analysis and/or identification of proteins and/or peptides by the following steps: labeling N-terminal peptides and/or proteins; performing tryptic digests of protein and/or peptide mixtures; purifying and/or identifying N-terminal peptides and/or proteins.

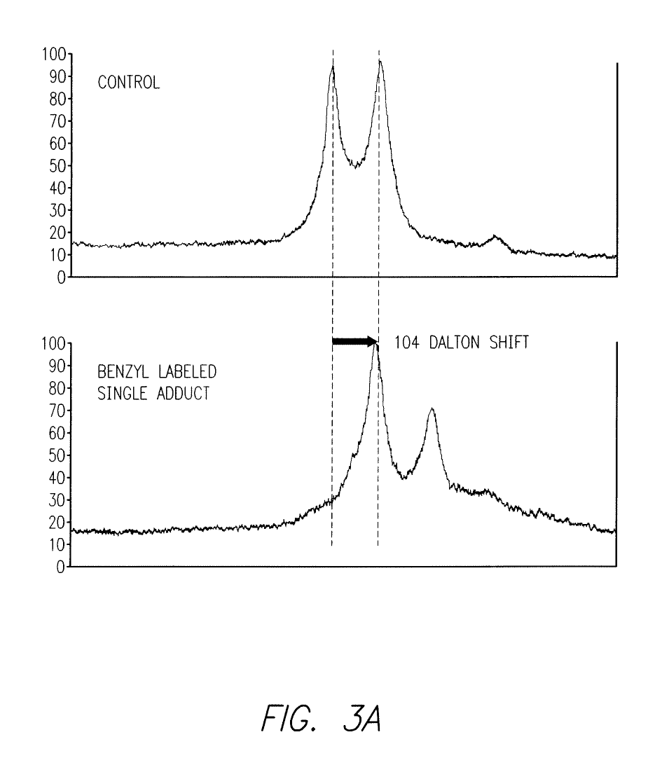

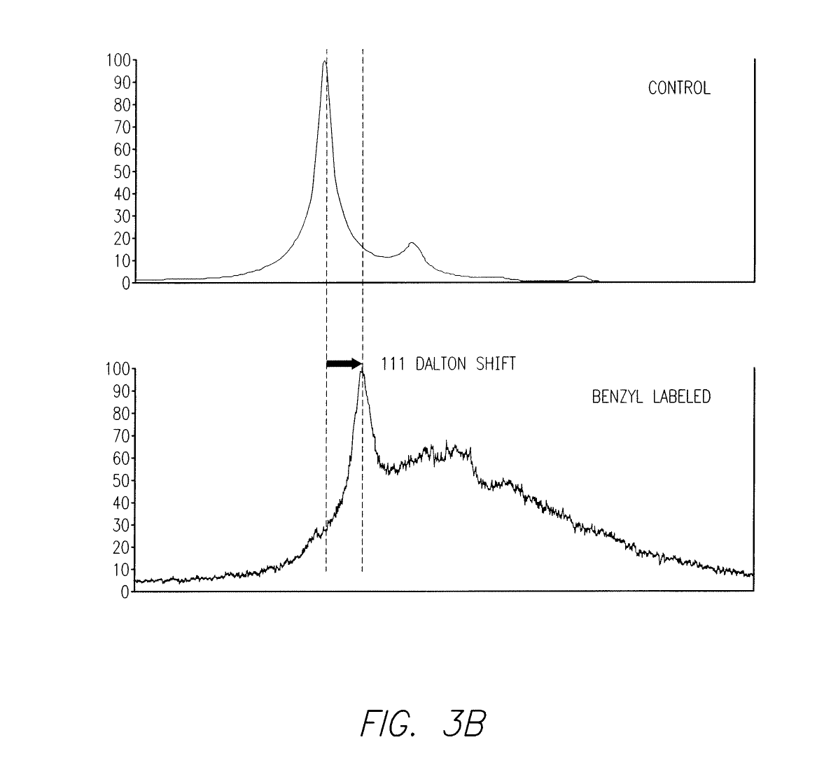

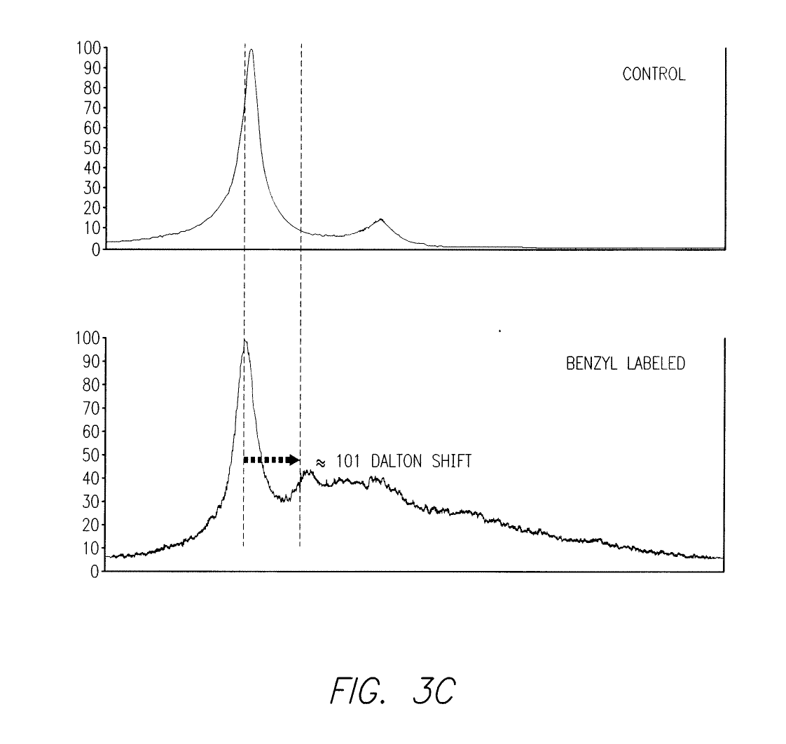

In another embodiment, the N-terminus of peptides and/or proteins are labeled by N-terminal mass defect tagging with biotinylated photo and/or acid/base cleavable mass defect aminooxy label. Results in accordance with this embodiment can been observed in FIGS. 3A, 3B and 3C.

In another embodiment, the N-terminus of peptides and/or proteins are labeled by N-terminal isotope modification for differential quantitation of heavy and light versions of aminooxy labels.

In another embodiment, the peptides and/or proteins are digested on amine-modified, trypic indigestible avidin, streptavidin, and/or neutravidin bound to solid support such as agarose.

In another embodiment, N-terminal peptides and/or proteins are identified by mass spectrometry and/or sequencing.

In another embodiment, the proteins and/or peptides are immobilized on a chromatography resin and/or array.

In another embodiment, the present subject matter is also directed at a kit intended for, but in no way limited to, (1) identification of a peptide and/or protein, (2) labeling of a peptide and/or protein N-termini, and/or (3) labeling of a peptide and/or protein with atoms with significant mass defect. The kit is useful for practicing the inventive methods disclosed herein. The kit is an assemblage of materials or components, including at least one of the inventive compositions. Thus, in some embodiments the kit contains a component including an N-terminus label, digest enzyme, label affinity column, mass defect atom, and combinations thereof.

The kits may include instructions for use. “Instructions for use” typically include a tangible expression describing the technique to be employed in using the components of the kit to effect a desired outcome, such as to conjugate a molecule to a peptide and/or protein.

The materials or components assembled in the kit can be provided to the practitioner stored in any convenient and suitable ways that preserve their operability, sterility and/or utility. The components are typically contained in suitable packaging material(s). As employed herein, the phrase “packaging material” refers to one or more physical structures used to house the contents of the kit, such as inventive components and the like. The packaging material is constructed by well known methods, preferably to provide a sterile, contaminant-free environment. The packaging materials employed in the kit are those customarily utilized for medical instruments. As used herein, the term “package” refers to a suitable solid matrix or material such as glass, plastic, paper, foil, and the like, capable of holding the individual kit components. Thus, for example, a package can be a plastic wrap used to contain components of the inventive subject matter. The packaging material generally has an external label which indicates the contents and/or purpose of the kit and/or its components.

By using the inventive methods, a number of applications have been demonstrated including: (1) high throughput proteomic analysis and identification of proteins and/or peptides by identification of N-Terminal peptides from tryptic digests of simple and complex protein and/or peptide mixtures (e.g., cell lysates, cell surface proteins, and plasma proteins) by N-Terminal specific peptide selection/isolation with or without further enrichment; (2) proteomic analysis of simple and complex protein and/or peptide mixtures by N-Terminal mass defect tagging with biotinylated photo- or acid/base cleavable mass defect aminooxy label; (3) proteomic analysis of simple and complex mixtures by N-Terminal isotope modification for differential quantitation with heavy and light versions of aminooxy labels; (4) N-Terminal amine specific labeling of cell surface with fluorophore or quantum dot for uv/visible/IR methods of cell and/or analyte detection and quantification, reaction and cell growth and/or viability quality control, and possible applications to flow cytometry; (5) high throughput determination of cell surface protein identity, quantity, topology, degradation and turnover; and (6) identification of N-Terminal peptides arising from simple and complex mixtures of proteins and/or peptides via N-Terminal biotinylation by the chemical method described above and purification and subsequent digestion on amine-modified, tryptic indigestible avidin, streptavidin, and neutravidin bound to a solid support (e.g., agarose). Another application includes modification of solid support immobilized proteins/peptides (e.g., chromatography resins and arrays) for any of the purposes above.

### EXAMPLES

The following examples are provided to better illustrate the claimed subject matter and are not to be interpreted as limiting the scope of the subject matter. To the extent that specific materials are mentioned, it is merely for purposes of illustration and is not intended to limit the subject matter. One skilled in the art may develop equivalent means or reactants without the exercise of inventive capacity and without departing from the scope of the subject matter.

### Example

### General Labeling Reaction

The affinity labeling protocol is a labeling reaction, followed by digestion and then chromatography to distinguish N-terminal peptides, allowing isolation and characterization of one peptide per digested protein. The reduced sample complexity facilitates protein sequencing and protein identification.

This process is a near-quantitative two-step (or three-step) reaction sequence, the result of which allows an arbitrary molecule to be conjugated to the N-termini of proteins either on live cells, on their own or in complex mixtures. The first step is the transamination and transformation of a peptide or protein n-terminal alpha-amine to an alpha-ketoamide (also called 2-oxo acyl group). This reactive intermediate is then condensed with an alkoxyamine (also called aminooxy) to form an oxime. The oxime can be further stabilized by reduction to an amine with sodium cyanoborohydride.

### Example 2

### Specific Labeling Reaction

Pyridoxal-5-phosphate and copper/glyoxylate mediated transamination of N-terminal (alpha-amine transformation to alpha-ketoamide) is followed by oxime formation via reaction with alkoxyamine (also known as hydroxylamine and aminooxy) reagents (subsequent reduction of oxime to secondary amine with sodium cyanoborohydride to stabilize conjugate in some cases) for the site-specific (N-terminal) labeling of proteins/peptides in vivo and in vitro.

### Example 3

### Experiment Design for Simple Mixture Modification

Mass spectrometry measures mass of analyte. Labeling proteins add mass to analyte per adduct. Single adduct adds mass to analyte per adduct. Single adduct adds mass equal to one label. Multiple adducts add mass equal to multiple labels. Thus, analysis by MALDI-MS of the protein mixture ensures that there is one label per protein.

### Example 4

### Experiment Design for Peptide Modification

The protein is N-terminally labeled and digested with trypsin. Mass spectrometry sequencing methods are used to validate that the label is on the N-terminal peptide.

### Example 5

### Experiment Design for Complex Mixture Modification

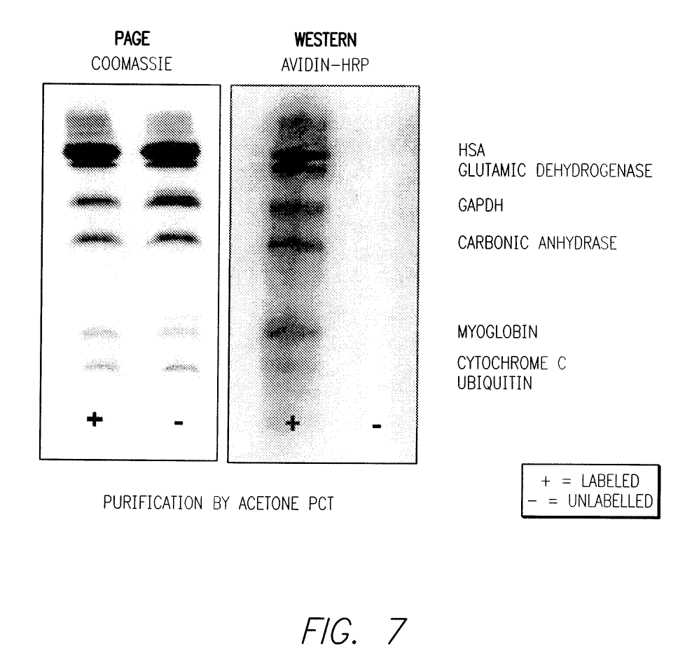

N-terminally label protein with biotin and run PAGE. Stain proteins with coomassie blue. Run Western Blot with avidin-hrp. Analyze PAGE and Western Blot results to ensure biotinylation of mixture of proteins. Results in accordance with this example can be seen in FIG. 7

### Example 6

### Advantages of the Development of N-Terminal Labeling Strategies

Upon the development of N-terminal labeling strategies, the subject matter increases performance of proteomic analysis and improves downstream processing of clinically relevant samples. The subject matter isolates N-terminal peptides from unlabelled peptides, lipids, small molecules and cellular debris by affinity chromatography. The subject matter also reduces sample complexity of typical bottom-up mass spectrometry by 50 fold.

The foregoing descriptions and examples of various embodiments of the subject matter known to the applicant at the time of filing this application have been presented and are intended for the purposes of illustration and description. The present descriptions and examples are not intended to be exhaustive nor limit the subject matter to the precise form disclosed and many modifications and variations are possible in light of the above teachings. The embodiments described serve to explain the principles of the subject matter and its practical application and to enable others skilled in the art to utilize the subject matter in various embodiments and with various modifications as are suited to the particular use contemplated. Therefore, it is intended that the subject matter disclosed herein not be limited to the particular embodiments disclosed.

While particular embodiments of the present subject matter have been shown and described, it will be obvious to those skilled in the art that, based upon the teachings herein, changes and modifications may be made without departing from this subject matter and its broader aspects and, therefore, the appended claims are to encompass within their scope all such changes and modifications as are within the true spirit and scope of this subject matter. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method for protein or peptide identification comprising:
adding a label to the N-terminus of a protein;
cleaving the protein into peptides using an enzyme whereby labeled peptides and non-labeled peptides are generated;
separating the labeled peptide from non-labeled peptides;
identifying the labeled peptide using mass spectrometry; and
matching the identified peptide to a protein.

2. The method of claim 1, wherein adding a label to the N-terminus of the protein comprises: transaminating and transforming the protein N-terminal alpha-amine to an alpha-ketoamide; reacting the N-terminal alpha-ketoamide with alkoxyamine to form an oxime; and attaching the label to the oxime.

3. The method of claim 2, wherein transamination of the N-terminal of the protein is accomplished by pyridoxal-5-phosphate and copper/gloxylate.

4. The method of claim 1, wherein adding a label to the N-terminus of the protein comprises: transaminating and transforming the protein N-terminal alpha-amine to an alpha-ketoamide; reacting the N-terminal alpha-ketoamide with alkoxyamine to form an oxime; reducing the oxime to a secondary amine with sodium cyanoborohydride; and attaching the label to the secondary amine.

5. The method of claim 4, wherein transamination of the N-terminal of the protein is accomplished by pyridoxal-5-phosphate and copper/gloxylate.

6. The method of claim 1, wherein adding a label to the N-terminus of the protein comprises: transaminating the N-terminal of the protein; transforming the transaminated protein to an alpha-ketoamide; and condensing the alpha-ketoamide with an alkoxyamine to form an oxime.

7. The method of claim 5, wherein the oxime is reduced to a secondary amine with sodium cyanoborohydride.

8. The method of claim 1, wherein adding a label to the N-terminus of the protein comprises: transaminating and transforming the protein N-terminal alpha-amine to an alpha-ketoamide; biotinylating the N-terminal alpha-ketoamide with aminoxy biotin to attach the biotin label.

9. The method of claim 8, wherein transamination of the N-terminal of the protein is accomplished by pyridoxal-5-phosphate and copper/glyoxylate

10. The method of claim 1, wherein the labeled peptide is separated from non-labeled peptides by liquid chromatography.

11. The method of claim 1, wherein the labeled peptide is separated from non-labeled peptides by gel electrophoresis.

12. The method of claim 1, wherein the labeled peptide is separated from non-labeled peptides by sodium dodecyl sulfate polyacrylamide gel electrophoresis (SDS-PAGE).

13. The method of claim 1, wherein the labeled peptide is identified by mass spectrometry, such as MALDI-TOF or ESI-TOF.

14. The method of claim 1, wherein the identified peptide is matched to a protein by computational analysis.

15. The method of claim 1, wherein the protein is purified from a protein mixture prior to adding a label to the N-terminus.

16. The method of claim 15, wherein the process for purifying the protein from the protein mixture is chosen from the group consisting of liquid chromatography, gel electrophoresis, and sodium dodecyl sulfate polyacrylamide gel electrophoresis (SDS-PAGE).

17. The method of claim 1, wherein the label added to the N-terminus of the protein comprises a unified atomic mass greater than 0.08.

18. A system for identifying a protein or peptide by mass spectrometry, the system comprising:
means for adding a label to the N-terminal of a protein by chemical means comprising: transaminating and transforming the protein N-terminal alpha-amine to an alpha-ketoamide, reacting the N-terminal alpha-ketoamide with alkoxyamine to form an oxime, and attaching the label to the oxime;
means for cleaving the protein into peptides whereby labeled peptides and non-labeled peptides are generated;
a tool for separating the labeled peptides from the non-labeled peptides selected from the group consisting of liquid chromatography, gel electrophoresis, and sodium dodecyl sulfate polyacrylamide gel electrophoresis (SDS-PAGE);
means for identifying the labeled peptide; and
means for computationally matching the identified peptide to a protein.

19. A kit for identifying a protein or peptide, comprising:
a label for attaching to the N-terminal of a protein or peptide;
one or more chemical reagents for the reaction of adding the label to the N-terminus of the protein;
one or more enzymes for cleaving the protein or peptide whereby labeled peptides and non-labeled peptides are generated;
one or more chemical reagents for the reaction of separating the labeled peptide from non-labeled peptides;
a device for identifying the labeled peptide; and
means for matching the identified peptide to a protein.
